(19) United States
(12) Patent Application Publication
(10) Pub. No.: US 2009/0094720 A1
(43) Pub. Date: Apr. 16, 2009

(54) Helmet mounted eye protectors with deployable eye shields

(76) Inventors: Thomas H. Stachler, Dayton, OH (US); Mary I. Grilliot, Dayton, OH (US); William L. Grilliot, Dayton, OH (US); Roy Paulson, Temecula, CA (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/974,654

(22) Filed: Oct. 15, 2007

(51) Int. Cl. A42B 3/00; A61F 9/00; A61F 9/02

(52) U.S. Cl. 2/5; 2/10; 2/453; 2/8.1

(57) ABSTRACT

An eye protector (16) is provided for use in connection with a protective helmet (10) worn by a firefighter or other emergency worker. The protective helmet (10) has a crown (12) and a brim (14) which projects forwardly and laterally from a lower edge of the crown (12). The eye protector (16) includes a pair of transparent, shatter-resistant eye shields (18) that are mounted to the underside of the brim (14) for movement between a storage position and a usage position. Each of the eye shields (18) may have a width W of at least 5.147 inches, may be manufactured from a coated polycarbonate sheet material, and may include an edge enhancement (29) to limit heat related deformation of the eye shield.

### CROSS-REFERENCE TO RELATED APPLICATIONS

Not Applicable.

### FEDERALLY SPONSORED RESEARCH OR DEVELOPMENT

Not Applicable.

### MICROFICHE/COPYRIGHT REFERENCE

Not Applicable.

### FIELD OF THE INVENTION

This invention pertains to eye protectors for use with a helmet for a firefighter or other emergency rescue worker.

### BACKGROUND OF THE INVENTION

Commonly, a helmet for a firefighter or for an industrial, chemical, or emergency rescue worker has a crown and a brim, which projects forwardly from the crown and which may project in other directions from the crown. Commonly, the helmet is equipped with a pair of eye shields, one for each eye of a wearer, and each of the pair of eye shields is adapted to be manually moved between a storage position and a usage position. In the usage position, but not in the storage position, the eye shield projects downwardly so as to shield a given eye of a wearer against sparks, liquids, particles, and other objects striking the front of the eye shield.

An example of an eye shield, as described in the preceding paragraph, is disclosed in U.S. Pat. No. 3,383,155 to Lester T. Bourke. As disclosed in U.S. Pat. No. 3,383,155, the disclosure of which is incorporated herein by reference, each of the pair of eye shields is mounted to a helmet, beneath a brim projecting forwardly from a crown of the helmet, and each of the pair of eye shields is adapted to be manually flipped between the storage and usage positions and is stable in either of the storage and usage positions. Similar eye shields are available commercially from various sources including Morning Pride Manufacturing, L.L.C. of Dayton, Ohio. While these shields have performed well for their intended purpose, there is always room for improvement.

### SUMMARY OF THE INVENTION

In accordance with one feature of the invention, an eye protector is provided mountable to a protective helmet for use by a firefighter or other emergency worker, the protective helmet having a brim. The eye protector includes a pair of eye shields mounted to the brim for movement between a storage position extending along the brim and a usage position extending downward from the brim to shield the eyes of a wearer, each of the eye shields having a width dimension of at least 5.147 inches.

According to one feature of the invention an eye protector is provided mountable to a protective helmet for use by a firefighter or other emergency worker, the protective helmet having a brim. The eye protector includes a pair of eye shields mounted to the brim for movement between a storage position extending along the brim and a usage position extending downward from the brim to shield the eyes of a wearer, and a pair of edge enhancements, each of the edge enhancements extending at least partially around a peripheral edge of each eye shield to limit heat related deformation of the eye shield.

As one feature, each of the eye shields is made from a coated polycarbonate sheet material.

In one feature, each of the edge enhancements extends over the entire peripheral edge of the corresponding eye shield.

According to one feature, the peripheral edge of each eye shield is defined by an upper edge connected to the brim, a lower edge opposite the upper edge, and two side edges extending from the upper edge to the lower edge, and each of the edge enhancements extends only over the lower edge of the corresponding eye shield.

In one feature, the peripheral edge of each eye shield is defined by an upper edge connected to the brim, a lower edge opposite the upper edge, and two side edges extending from the upper edge to the lower edge, and each of the edge enhancements extends over the lower edge and both of the side edges of the corresponding eye shield.

As one feature, the edge enhancements include a pair of support frames, and each of the support frames has a groove receiving an extended edge of the corresponding eye shield.

In one feature, the edge enhancements include a pair of support frames, and each of the support frames is riveted to a face surface of the corresponding eye shield.

As one feature, the edge enhancements include a thermal insulative material applied at least partially around a peripheral edge of each eye shield.

In accordance with one feature of the invention, an eye shield is provided mountable to a protective helmet for use by a firefighter or other emergency worker. The eye shield is mounted to a brim of the protective helmet for movement between a storage position extending along the brim and a usage position extending downward from the brim to shield the eyes of a wearer. The eye shield has a width dimension of at least 5.147 inches.

According to one feature of the invention, an eye shield is provided mountable to a protective helmet for use by a firefighter or other emergency worker. The eye shield is mounted to a brim of the protective helmet for movement between a storage position extending along the brim and a usage position extending downward from the brim to shield the eyes of a wearer. An edge enhancement extends at least partially around a peripheral edge of the eye shield to limit heat related deformation of the eye shield.

Other objects, features, and advantages of the invention will become apparent from a review of the entire specification, including the appended claims and drawings.

### BRIEF DESCRIPTION OF THE DRAWINGS

FIG. 1 is a fragmentary, pictorial view looking toward the front of a helmet equipped with a pair of eye shields embodying the invention, with the eye shield on the right showing an alternate embodiment to the eye shield on the left;

FIG. 2 is a fragmentary, pictorial view looking toward the underside of the helmet, with the pair of eye shields in a storage position;

FIG. 3 is a view similar to FIG. 1 but with the left and right eye shields illustrating respective alternate embodiments for the inventions;

FIGS. 4A, 4B, and 4C are views taken along line 4-4 in FIG. 1, showing respective alternate embodiments of an edge enhancement of the invention; and

FIG. 5 is a view similar to FIGS. 4A-4C, but showing another alternate embodiment of an edge enhancement of the invention.

### DETAILED DESCRIPTION OF THE PREFERRED EMBODIMENT

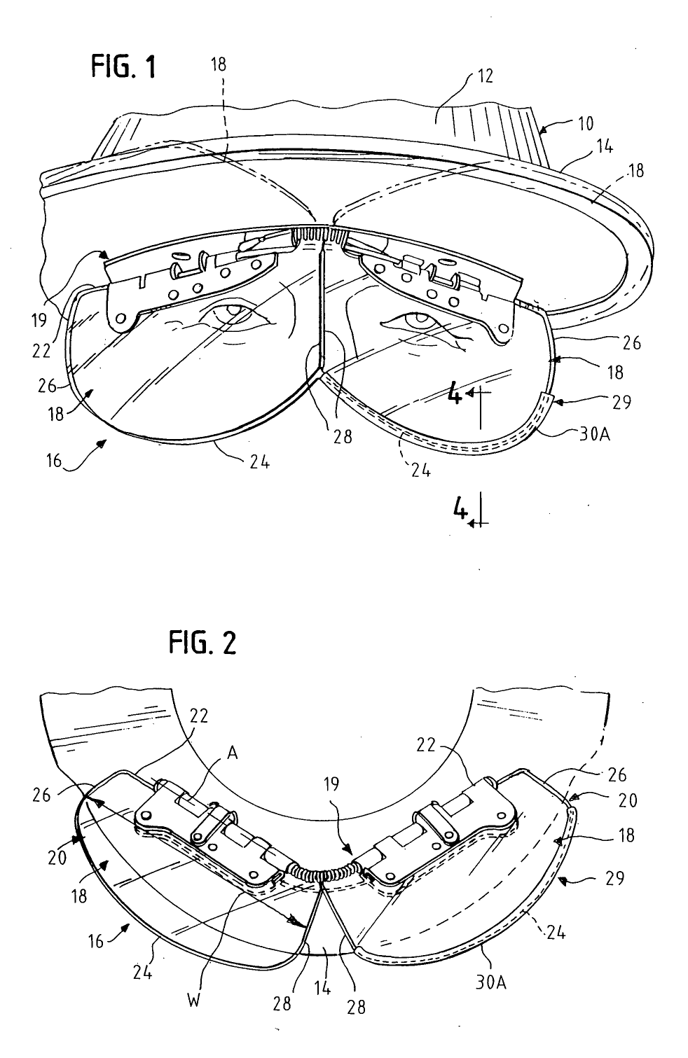

As illustrated in FIG. 1, a protective helmet 10 worn by a firefighter has a crown 12 and a brim 14 which projects forwardly and laterally from a lower edge of the crown 12. The helmet 10 is equipped with an eye protector 16 in the form of a pair of transparent, shatter-resistant eye shields 18. The shields 18 are mounted to the underside of the brim 14 where the brim 14 project forwardly, via hinges, springs, and other elements, shown generally at 19, as disclosed in U.S. Pat. No. 3,383,155, supra, so as to be manually flippable between a storage position and a usage position. FIG. 1 shows the shields 18 in the usage position, with each shield 18 extending downward from the brim to shield the eyes of a wearer against sparks, liquids, particles, and other objects striking the front of the shield 18. The storage position is shown in phantom in FIG. 1, with each shield 18 projecting forwardly, along and beneath the brim 14.

As best seen in FIG. 2 each of the shields 18 has a peripheral edge 20 defined by an upper edge 22, a lower edge 24 opposite the upper edge 22, and two side edges 26 and 28 extending from the upper edge 22 to the lower edge 24. Each of the upper edges 22 is connected to the brim 14 by the mount hardware 19 for movement of the shield 18 between the storage position and the usage position.

Each of the shields 18 has a width W extending from one side edge 26 to the other side edge 28, parallel to the hinge axis A, and it is preferred that W be at least 5.147 inches to provide increased protection for the eye from projectiles and other airborne object over conventional shields. It has been found that by making the width W at least 5.147 inches, the shields 18 can meet the resistance to impact tests required for certification under National Fire Protection Association NFPA 1971, 2007 edition.

It is also preferred that each of the shields 18 be made from a coated polycarbonate sheet material, one example of which is a coated bisphenol. A polycarbonate sheet material sold under the trademark MAKROLON AR by Sheffield Plastics Inc., A Bayer MaterialScience Company, Sheffield, Mass., and identified as material number SH005369. It has been found that such material is superior in meeting the requirements under NFPA 1971, 2007 edition.

As best seen by the shield 18 illustrated on the left side of FIG. 1, the peripheral edge 20 of each of the lenses 18 can be exposed as is conventional, with no structural support being provided other than the mount hardware 19. This embodiment provides for the simplest construction of the eye protector 16.

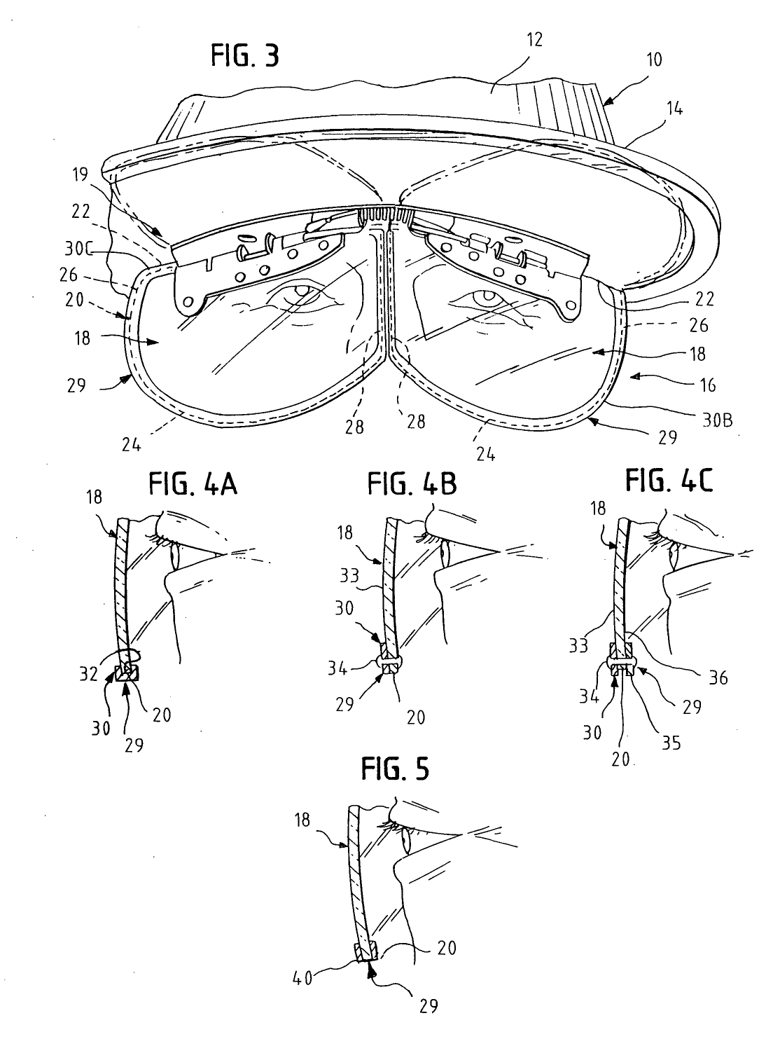

As best seen by the shield 18 illustrated on the right side of FIG. 1 and by the shields 18 illustrated in FIG. 3, in some embodiments it is preferred that an edge enhancement 29 be provided in the form of a structural support frame 30 extend at least partially around the peripheral edge 20 of each eye shield 18 to limit heat related deformation of the eye shield 18 such as may occur when the firefighter or other emergency worker is in a fire environment. Preferably, the frames 30 are made out of a suitable metal or a suitable high temperature plastic or composite. The frames 30 provide additional structural integrity for each of the eye shields 18. In this regard, it has been found that such support frames 30 provide additional assurance that the shields 18 can meet the certification requirements under NFPA 1971, 2007 Edition, particularly the thermal requirements under the standard. Furthermore, it is believed that such frames can allow for the transparent portion of the shields 18 to be manufactured from a larger variety of materials, with a larger variety of material thicknesses for those materials, while still allowing the eye protector 16 to satisfy the requirements for certification under NFPA 1971, 2007 Edition.

As shown by the various embodiments of FIGS. 1 and 3, there are a number of possibilities for the construction of the frames 30. More specifically, in the embodiment shown on the lens 18 on the right side of FIG. 1, the frame 30A extends only over the lower edge 24 of each of the shields 18. In the embodiment shown on the right side in FIG. 3, the frame 30B extends over the lower edge 24 and both of the side edges 26 and 28. Finally, for the embodiment shown on the left side of FIG. 3, the frame 30C extends around the entire peripheral edge 20 of each of the eye shields 18. The particular type of frame 30A, 30B and 30C selected will be highly dependent upon the particular material and/or material thickness selected for the transparent material of the shield 18.

With reference to FIGS. 4A-4C, it can be seen that there are a number of options for attaching the frames 30A-30C to the transparent material of the eye shield 18. As seen in FIG. 4A, the frame 30 can be provided with a groove 32 that receives the edge 20 of the eye shield 18 with either a close or interference fit, which can be further supplemented with a suitable bonding material desired. As seen in the embodiment of FIG. 4B, the frame 30 can be riveted to a face 33 of the eye shield 18 using a plurality of suitable rivets 34 (only one shown) that are spaced along the length of the frame 30 and the edge 20. In another option shown in FIG. 4C, the frame 30 can include an additional strip of structural support material 35 that is riveted to a back face 36 of the eye shield 18 for additional structure support. As yet another option, both of the frame configurations shown in FIGS. 4B and 4C can be bonded to the respective face 33,36 of the eye shield 18, rather than riveted, using any suitable bonding agent or material.

As another option, the edge enhancements 29 can be provided in the form of a thermal insulative material 40 that is applied at least partially around the peripheral edge 20 of each eye shield 18, as shown in FIG. 5. In this regard, the thermal insulative material 40 can extend around the peripheral edge 20 in the same fashion as the frames 30 shown in any of FIGS. 1, 2, and 3. In addition to the configuration shown in FIG. 5, the thermal insulative material 40 can be applied to just the front face 33 of the eye shield similar to the frame 30 shown in FIG. 4B, or to both the front and back faces 33 and 36 similar to the frame 30 shown in FIG. 4C. The thermal insulative material 40 can be any suitable material that will insulate the edge of the eye shield 18 against radiant, convective, and/or conductive heat transfer, and preferably is a material that will insulate against all three types of heat transfer. The material can be applied as a coating and/or spray, or can be a separate structural piece that is attached as previously described for the support frames 30. In this regard, it should be understood that the support frames 30 can also be made entirely from and/or coated with a suitable thermal insulative material as described above.

1. An eye protector mountable to a protective helmet for use by a firefighter or other emergency worker, the protective helmet having a brim, the eye protector comprising:
a pair of eye shields mounted to the brim for movement between a storage position extending along the brim and a usage position extending downward from the brim to shield the eyes of a wearer, each of the eye shields having a width dimension of at least 5.147 inches.

2. The eye protector of claim 1 wherein each of the eye shields is made from a coated polycarbonate sheet material to limit heat related deformation of the eye shield.

3. The eye protector of claim 1 further comprising a pair of edge enhancements, each of the edge enhancements extending at least partially around a peripheral edge of each eye shield.

4. The eye protector of claim 3 wherein each of the edge enhancements extends over the entire peripheral edge of the corresponding eye shield.

5. The eye protector of claim 3 wherein the peripheral edge of each eye shield is defined by an upper edge connected to the brim, a lower edge opposite the upper edge, and two side edges extending from the upper edge to the lower edge, and each of the edge enhancements extends only over the lower edge of the corresponding eye shield.

6. The eye protector of claim 3 wherein the peripheral edge of each eye shield is defined by an upper edge connected to the brim, a lower edge opposite the upper edge, and two side edges extending from the upper edge to the lower edge, and each of the edge enhancements extends over the lower edge and both of the side edges of the corresponding eye shield.

7. The eye protector of claim 3 wherein the edge enhancements comprise support frames, and each of the support frames has a groove receiving an extended edge of the corresponding eye shield.

8. The eye protector of claim 3 wherein edge enhancements comprise support frames, and each of the support frames is riveted or bonded to a face surface of the corresponding eye shield.

9. The eye protector of claim 3 wherein the edge enhancements comprise a thermal insulative material applied at least partially around a peripheral edge of each eye shield.

10. An improvement in an eye shield mountable to a protective helmet for use by a firefighter or other emergency worker, the eye shield mounted to a brim of the protective helmet for movement between a storage position extending along the brim and a usage position extending downward from the brim to shield the eyes of a wearer, the improvement wherein the eye shield has a width dimension of at least 5.147 inches.

11. The improvement of claim 10 wherein the eye shield is made from a coated polycarbonate sheet material.

12. The improvement of claim 10 further comprising an edge enhancement extending at least partially around a peripheral edge of the eye shield.

13. The improvement of claim 12 wherein the edge enhancement extends over the entire peripheral edge of the eye shield.

14. The improvement of claim 12 wherein the peripheral edge of the eye shield is defined by an upper edge connected to the brim, a lower edge opposite the upper edge, and two side edges extending from the upper edge to the lower edge, and the edge enhancement extends only over the lower edge of the eye shield.

15. The improvement claim 12 wherein the peripheral edge of the eye shield is defined by an upper edge connected to the brim, a lower edge opposite the upper edge, and two side edges extending from the upper edge to the lower edge, and the edge enhancement extends over the lower edge and both of the side edges of the corresponding eye shield.

16. The improvement of claim 12 wherein the edge enhancement comprises a support frame having a groove receiving an extended edge of the eye shield.

17. The improvement of claim 12 wherein the edge enhancement comprises a support frame is riveted to a face surface of the eye shield.

18. The improvement of claim 13 wherein the edge enhancements comprise a thermal insulative material applied at least partially around a peripheral edge of each eye shield.

19. An eye protector mountable to a protective helmet for use by a firefighter or other emergency worker, the protective helmet having a brim, the eye protector comprising:
a pair of eye shields mounted to the brim for movement between a storage position extending along the brim and a usage position extending downward from the brim to shield the eyes of a wearer; and
a pair of edge enhancements, each of the edge enhancements extending at least partially around a peripheral edge of each eye shield to limit heat related deformation of the eye shield.

20. The eye protector of claim 19 wherein each of the eye shields is made from a coated polycarbonate sheet material.

21. The eye protector of claim 19 wherein each of the edge enhancements extends over the entire peripheral edge of the corresponding eye shield.

22. The eye protector of claim 19 wherein the peripheral edge of each eye shield is defined by an upper edge connected to the brim, a lower edge opposite the upper edge, and two side edges extending from the upper edge to the lower edge, and each of the edge enhancements extends only over the lower edge of the corresponding eye shield.

23. The eye protector of claim 19 wherein the peripheral edge of each eye shield is defined by an upper edge connected to the brim, a lower edge opposite the upper edge, and two side edges extending from the upper edge to the lower edge, and each of the edge enhancements extends over the lower edge and both of the side edges of the corresponding eye shield.

24. The eye protector of claim 19 wherein the edge enhancements comprise support frames, and each of the support frames has a groove receiving an extended edge of the corresponding eye shield.

25. The eye protector of claim 19 wherein the edge enhancements comprise support frames, and each of the support frames is riveted or bonded to a face surface of the corresponding eye shield.

26. The eye protector of claim 19 wherein the edge enhancements comprise a thermal insulative material applied at least partially around a peripheral edge of each eye shield.

27. An improvement in an eye shield mountable to a protective helmet for use by a firefighter or other emergency worker, the eye shield mounted to a brim of the protective helmet for movement between a storage position extending along the brim and a usage position extending downward from the brim to shield the eyes of a wearer, the improvement wherein an edge enhancement extends at least partially around a peripheral edge of the eye shield to limit thermal related deformation of the eye shield to limit heat related deformation of the eye shield.

28. The improvement of claim 27 wherein the eye shield is made from a coated polycarbonate sheet material.

29. The improvement of claim 27 wherein the edge enhancement extends over the entire peripheral edge of the eye shield.

30. The improvement of claim 27 wherein the peripheral edge of the eye shield is defined by an upper edge connected to the brim, a lower edge opposite the upper edge, and two side edges extending from the upper edge to the lower edge, and the edge enhancement extends only over the lower edge of the eye shield.

31. The improvement claim 27 wherein the peripheral edge of the eye shield is defined by an upper edge connected to the brim, a lower edge opposite the upper edge, and two side edges extending from the upper edge to the lower edge, and the edge enhancement extends over the lower edge and both of the side edges of the corresponding eye shield.

32. The improvement of claim 27 wherein the edge enhancement comprises a support frame having a groove receiving an extended edge of the eye shield.

33. The improvement of claim 27 wherein the edge enhancement comprises a support frame riveted or bonded to a face surface of the eye shield.

34. The improvement of claim 27 wherein the edge enhancement comprises a thermal insulative material applied at least partially around a peripheral edge of each eye shield.
